(19) United States
(12) Patent Application Publication
(10) Pub. No.: US 2024/0071630 A1
(43) Pub. Date: Feb. 29, 2024

(54) METHOD AND APPARATUS FOR DETERMINING DRUG CODE, ELECTRONIC DEVICE, AND COMPUTER MEDIUM

(71) Applicants: Beijing Wodong Tianjun Information Technology Co., Ltd., Beijing (CN); Beijing Jingdong Century Trading Co., Ltd., Beijing (CN)

(72) Inventors: Nan ZHAO, Beijing (CN); Youzheng WU, Beijing (CN)

(73) Assignees: Beijing Wodong Tianjun Information Technology Co., Ltd., Beijing (CN); Beijing Jingdong Century Trading Co., Ltd., Beijing (CN)

(21) Appl. No.: 18/272,315

(22) PCT Filed: Dec. 15, 2021
(86) PCT No.: PCT/CN2021/138298

§ 371 Date: Jul. 13, 2023

(30) Foreign Application Priority Data
Jan. 15, 2021 (CN) 202110054078.1

(51) Int. Cl. G16H 70/40

(52) U.S. Cl. CPC G16H 70/40

(57) ABSTRACT

A method and apparatus for determining a drug code. The method may include: obtaining a specification text of a drug (201); extracting drug key information in the specification text (202); obtaining, on the basis of a pre-created code inverted index, at least one code related to the drug key information and an ingredient corresponding to each code (203); and screening the at least one code on the basis of the drug key information and the ingredient corresponding to each code to obtain an anatomical therapeutic chemical classification system code of the drug (204).

### CROSS-REFERENCE TO RELATED APPLICATIONS

This application is a U.S. National Stage of International Application No. PCT/CN2021/138298, filed on Dec. 15, 2021, which claims the priority from Chinese Patent Application No. 202110054078.1, filed on Jan. 15, 2021 and entitled “Method and Apparatus for Determining Drug Code, Electronic Device and Computer Medium,” the entire disclosure of which is hereby incorporated by reference.

### TECHNICAL FIELD

The present disclosure relates to the field of computer technology, specifically to the field of artificial intelligence technology, and particularly to a method and apparatus for determining a drug code, an electronic device, a computer readable medium and a computer program product.

### BACKGROUND

An anatomical therapeutic chemical classification system, abbreviated as ATC (anatomical therapeutic chemical) system, is the official classification system of the World Health Organization for drugs. With the development and progress of medical information systems, various levels of medical institutions, medical insurance administrations and medical insurance institutions have gradually established a precise medicine management system with an ATC code system as a basis.

### SUMMARY

Embodiments of the present disclosure propose a method and apparatus for determining a drug code, an electronic device, and a computer readable medium.

In a first aspect, an embodiment of the present disclosure provides a method for determining a drug code, including: acquiring a description text of a drug; extracting key information of the drug in the description text; obtaining at least one code related to the key information of the drug and an ingredient corresponding to each code based on a pre-created code inverted index; and screening the at least one code based on the key information of the drug and the ingredient corresponding to each code to obtain an anatomical therapeutic chemical classification system code of the drug.

In some embodiments, the key information of the drug includes a drug ingredient. Screening the at least one code based on the key information of the drug and the ingredient corresponding to each code to obtain the anatomical therapeutic chemical classification system code of the drug includes: detecting, for each code in the at least one code, whether an ingredient corresponding to the code satisfies one of a plurality of rules having a priority order, the plurality of rules being determined based on the drug ingredient; determining a preliminarily screened candidate code including the code, in response to determining that the ingredient corresponding to the code satisfies one of the plurality of rules and detections for all codes are completed; and determining that the preliminarily screened candidate code is the anatomical therapeutic chemical classification system code of the drug, in response to detecting that the preliminarily screened candidate code refers to only one code.

In some embodiments, the plurality of rules are arranged in a descending order of priorities as follows: (1) the ingredient corresponding to the code includes all drug ingredients of the drug when there are two or more kinds of drug ingredients; (2) the ingredient corresponding to the code includes at least one drug ingredient of the drug and contains a word “compound” when there are two or more kinds of drug ingredients; (3) the ingredient corresponding to the code includes at least one drug ingredient of the drug and does not contain the word “compound” when there are two or more kinds of drug ingredients; and (4) the ingredient corresponding to the code includes the drug ingredient when there is one kind of drug ingredient.

In some embodiments, the key information of the drug includes a drug ingredient. Screening the at least one code based on the key information of the drug and the ingredient corresponding to each code to obtain the anatomical therapeutic chemical classification system code of the drug includes: detecting, for each code in the at least one code, whether an ingredient corresponding to the code matches the drug ingredient; obtaining a preliminarily screened candidate code including the code, in response to determining that the ingredient corresponding to the code matches the drug ingredient and detections for all codes are completed; and determining that the preliminarily screened candidate code is the anatomical therapeutic chemical classification system code of the drug, in response to detecting that the preliminarily screened candidate code refers to only one code.

In some embodiments, the key information of the drug further includes a drug indication, and screening the at least one code based on the key information of the drug and the ingredient corresponding to each code to obtain the anatomical therapeutic chemical classification system code of the drug further includes: determining a disease type corresponding to the drug based on the drug indication, in response to detecting that the preliminarily screened candidate code refers to a plurality of codes; and screening a code corresponding to the disease type from the preliminarily screened candidate code as the anatomical therapeutic chemical classification system code of the drug.

In some embodiments, determining the disease type corresponding to the drug based on the drug indication includes: performing a disease classification on the indication using a pre-trained classification model to obtain the disease type outputted by the classification model.

In a second aspect, an embodiment of the present disclosure provides an apparatus for determining a drug code, including: an acquiring unit, configured to acquire a description text of a drug; an extracting unit, configured to extract key information of the drug in the description text; an obtaining unit, configured to obtain at least one code related to the key information of the drug and an ingredient corresponding to each code based on a pre-created code inverted index; and a screening unit, configured to screen the at least one code based on the key information of the drug and the ingredient corresponding to each code to obtain an anatomical therapeutic chemical classification system code of the drug.

In some embodiments, the key information of the drug includes a drug ingredient, and the screening unit includes: a detecting module, configured to detect, for each code in the at least one code, whether an ingredient corresponding to the code satisfies one of a plurality of rules having a priority order, the plurality of rules being determined based on the drug ingredient; a preliminarily screening module, configured to determine a preliminarily screened candidate code including the code, in response to determining that the ingredient corresponding to the code satisfies one of the plurality of rules and detections for all codes are completed; and a determining module, configured to determine that the preliminarily screened candidate code is the anatomical therapeutic chemical classification system code of the drug, in response to detecting that the preliminarily screened candidate code refers to only one code.

In some embodiments, the plurality of rules are arranged in a descending order of priorities as follows: (1) the ingredient corresponding to the code includes all drug ingredients of the drug when there are two or more kinds of drug ingredients; (2) the ingredient corresponding to the code includes at least one drug ingredient of the drug and contains a word “compound” when there are two or more kinds of drug ingredients; (3) the ingredient corresponding to the code includes at least one drug ingredient of the drug and does not contain the word “compound” when there are two or more kinds of drug ingredients; and (4) the ingredient corresponding to the code includes the drug ingredient when there is one kind of drug ingredient.

In some embodiments, the key information of the drug includes a drug ingredient. the screening unit includes: a matching module configured to detect, for each code in the at least one code, whether an ingredient corresponding to the code matches the drug ingredient; a responding module configured to obtain a preliminarily screened candidate code including the code, in response to determining that the ingredient corresponding to the code matches the drug ingredient and detections for all codes are completed; and an encoding module configured to determine that the preliminarily screened candidate code is the anatomical therapeutic chemical classification system code of the drug, in response to detecting that the preliminarily screened candidate code refers to only one code.

In some embodiments, the key information of the drug further includes a drug indication. The screening unit further includes: a classifying module configured to determine a disease type corresponding to the drug based on the drug indication, in response to detecting that the preliminarily screened candidate code refers to a plurality of codes; and a confirming module configured to screen a code corresponding to the disease type from the preliminarily screened candidate code as the anatomical therapeutic chemical classification system code of the drug.

In some embodiments, the classifying module may be further configured to performing a disease classification on the indication using a pre-trained classification model to obtain the disease type outputted by the classification model.

In a third aspect, an embodiment of the present disclosure provides an electronic device, including: one or more processors; and a storage apparatus, storing one or more programs. The one or more programs, when executed by the one or more processors, cause the one or more processors to implement the method according to any implementation in the first aspect.

In a fourth aspect, an embodiment of the present disclosure provides a computer readable medium, storing a computer program. The program, when executed by a processor, implements the method according to any implementation in the first aspect.

### BRIEF DESCRIPTION OF THE DRAWINGS

After reading detailed descriptions of non-limiting embodiments given with reference to the following accompanying drawings, other features, objectives and advantages of the present disclosure will be more apparent.

FIG. 1 is a diagram of an exemplary system architecture in which an embodiment of the present disclosure may be applied;

FIG. 2 is a flowchart of an embodiment of a method for determining a drug code according to the present disclosure;

FIG. 3 is a flowchart of an embodiment of a method for obtaining an anatomical therapeutic chemical classification system code of a drug according to the present disclosure;

FIG. 4 is a flowchart of another embodiment of the method for obtaining an anatomical therapeutic chemical classification system code of a drug according to the present disclosure;

FIG. 5 is a schematic structural diagram of an embodiment of an apparatus for determining a drug code according to the present disclosure; and

FIG. 6 is a schematic structural diagram of an electronic device adapted to implement embodiments of the present disclosure.

### DETAILED DESCRIPTION OF EMBODIMENTS

The present disclosure is further described below in detail by combining the accompanying drawings and the embodiments. It may be appreciated that the specific embodiments described herein are merely used for explaining the relevant disclosure, rather than limiting the disclosure. In addition, it should be noted that, for ease of description, only the parts related to the relevant disclosure are shown in the accompanying drawings.

It should be noted that the embodiments in the present disclosure and the features in the embodiments may be combined with each other on a non-conflict basis. The present disclosure will be described below in detail with reference to the accompanying drawings and in combination with the embodiments.

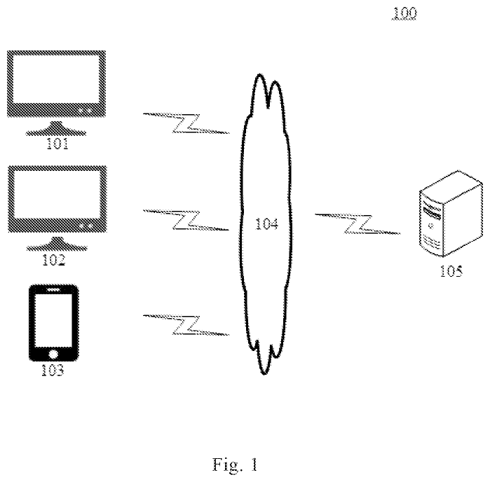

FIG. 1 illustrates an exemplary system architecture 100 in which a method for determining a drug code according to the present disclosure may be applied.

As shown in FIG. 1, the system architecture 100 may include terminal devices 101, 102 and 103, a network 104 and a server 105. The network 104 serves as a medium providing a communication link between the terminal devices 101, 102 and 103 and the server 105. The network 104 may include various types of connections, and generally include a wireless communication link.

The terminal devices 101, 102 and 103 may interact with the server 105 via the network 104, to receive or send a message, etc. Various communication client applications (e.g., an instant messaging tool, and an email client) may be installed on the terminal devices 101, 102 and 103.

The terminal devices 101, 102 and 103 may be hardware or software. When being the hardware, the terminal devices 101, 102 and 103 may be user devices having communication and control functions, and the above user devices may communicate with the server 105. When being the software, the terminal devices 101, 102 and 103 may be installed in the above user devices. The terminal devices 101, 102 and 103 may be implemented as a plurality of pieces of software or a plurality of software modules (e.g., software or software modules for providing a distributed service), or as a single piece of software or a single software module, which will not be specifically defined here.

The server 105 may be a server providing various services, for example, a backend server that determines a drug code and provides support to a drug processing system on the terminal devices 101, 102 and 103. The backend server may perform analytic processing on a description text of a drug in a network, and feed back a processing result (e.g., a determined ATC code) to the terminal devices.

It should be noted that the server may be hardware or software. When being the hardware, the server may be implemented as a distributed server cluster composed of a plurality of servers, or as a single server. When being the software, the server may be implemented as a plurality of pieces of software or a plurality of software modules (e.g., software or software modules for providing a distributed service), or as a single piece of software or a single software module, which will not be specifically defined here.

It should be noted that the method for determining a drug code provided by the embodiments of the present disclosure is generally performed by the server 105.

It should be appreciated that the numbers of the terminal devices, the networks and the servers in FIG. 1 are merely illustrative. Any number of terminal devices, networks and servers may be provided based on actual requirements.

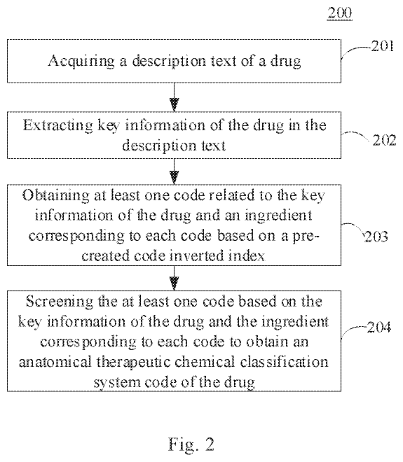

In some alternative implementations of this embodiment, as shown in FIG. 2, FIG. 2 illustrates a flow 200 of an embodiment of a method for determining a drug code according to the present disclosure. The method for determining a drug code includes the following steps.

Step 201, acquiring a description text of a drug.

In this embodiment, the description of the drug refers to a legal document that states the important information of the drug, and is a legal guide for selecting the drug. Accurately reading and understanding the description before administration is a prerequisite for safe administration. The description of the drug includes the name, specification, manufacturing enterprise, expiry date, usage, dosage, drug ingredient, indication or major function, contraindication, adverse reaction and precautions of the drug. Here, the product name of the drug includes a generic name, a brand name, an English name, a chemical name, and the like. Generally, as long as a user can be aware of the generic name of the drug, the repeated medication can be avoided. The description text of the drug is a text used to denote the content of the description of the drug.

An executing body on which the method for determining a drug code runs may obtain the description text by various means, for example, obtain the description text from a terminal in real time or read the description text from a memory, which is not limited in this embodiment.

Step 202, extracting key information of the drug in the description text.

In this embodiment, by acquiring the description text of the drug, natural language processing can be performed on the description text to obtain the key information of the drug. The key information of the drug includes a drug ingredient or information related to the drug ingredient. The information related to the drug ingredient includes the product name of the drug, the indication or major function of the drug, the contraindication, the adverse reaction, and the like. In this embodiment, the drug ingredient may alternatively be the main ingredient of the drug.

At present, the natural language processing technology has been widely applied in the scenarios in life where semantic understanding is required. For example, an entity recognition technique may recognize an entity (e.g., a drug name, a disease name, and a treatment method) in a piece of text. In this way, content such as a diagnosis and a prescription in a doctor's order can be automatically analyzed, and the medical information management can be performed in a structured manner. For example, a text classification technique can be applied to an intelligent triage scenario to intelligently analyze the description of the disease of a patient, and a consulting room is precisely matched based on the description of the disease, thereby improving the triage efficiency. The combination of the natural language processing technology and the medical scenario can improve the intelligentization of the medical scenario, thereby providing better experience for the user.

In this embodiment, the drug ingredient, the product name of the drug, the indication or major function of the drug, the contraindication, the adverse reaction, etc. in the description text can be extracted by natural language processing. In the description of the drug, the drug ingredient is generally included in the natural language described in a short piece of text, for example, “this product is a compound preparation, containing 10 mg of clindamycin hydrochloride (calculated as clindamycin) and 8 mg of metronidazole per ml. Excipients are: glycerin and ethanol”. By means of a natural language processing model (e.g., a named entity recognition model), the main ingredients therein (non-auxiliary ingredients or excipients) can be extracted. For the above description text, the drug ingredient extracted by the natural language processing model includes clindamycin hydrochloride, metronidazole, glycerin and ethanol.

Alternatively, a natural language model composed of BERT (Bidirectional Encoder Representation from Transformers) and CRF (conditional random field) may be adopted to perform training, thus obtaining a trained named entity recognition model which performs entity recognition on a drug ingredient. The key information of the drug is obtained through the named entity recognition model. In practice, the accuracy of the recognition result of the named entity recognition model can be close to 90%, which can completely meet the actual clinical use requirement.

Depending on the nature and characteristics of the drug, the drug includes a compound drug and a single drug. The single drug refers to a single prescription preparation, and mainly includes one kind of drug ingredient. The compound drug refers to a mixed preparation of two or more medicines, which may be a mixture of traditional Chinese medicines, a mixture of western medicines, or a mixture of a traditional Chinese medicine and a western medicine. The compound drug contains two or more drug ingredients. In this embodiment, for the different kinds of drugs described above, the drug ingredient in the key information of the drug may refer to one or more kinds of drug ingredients.

Step 203, obtaining at least one code related to the key information of the drug and an ingredient corresponding to each code based on a pre-created code inverted index.

In this embodiment, the code inverted index is an index library created before the key information of the drug in the description text is extracted. The code inverted index only needs to be created once, and then can be reused.

In this embodiment, the created code inverted index is determined based on the code of the drug that is required to be determined. The method for determining a drug code provided in the present disclosure is used to determine the ATC code of the drug. Therefore, the code inverted index may refer to that inverted indexing is performed on the classification information (ATC Chinese names, ATC English names and ATC codes) of the ATC code classification standards defined by the World Health Organization by groups, for example, a code inverted index as shown in Table 1. In Table 1, there are ATC codes and the Chinese names and English names of the chemical substances corresponding to the ATC codes. Here, a chemical substance is also a drug ingredient, that is, the drug ingredient corresponding to the ATC code. For example, “ [symbol] [symbol] ” has a corresponding English name “ticlatone” and a corresponding ATC code “D01AE08.”

It should be noted that there may be one or a plurality of drug ingredients, and thus, for the plurality of drug ingredients, there must be a plurality of corresponding ATC codes, and for the one drug ingredient, there may be a plurality of corresponding ATC codes. For example, in Table 1, the ATC codes to which the ATC codes of medicines containing “tegafur” may correspond include: “L01BC03” and “L01BC53.”

TABLE 1

| Chinese name | English name | ATC code |
|---|---|---|
| [symbol] | ticlatone | D01AE08 |
| [symbol] | teclozan | P01AC04 |
| [symbol] | tilidine | N02AX01 |
| [symbol] ( [symbol] ) | tegafur | L01BC03 |
| [symbol] , [symbol] | Tegafur, combinations | L01BC53 |
| . . . | . . . | . . . |

In practical application scenarios, search engine software (e.g., Elasticsearch) may be used to complete the indexing for an ATC code defined by the World Health Organization. By establishing an inverted index search engine, when a corresponding text field is searched, for example, classification information containing a certain field (e.g., metronidazole) in an ATC Chinese name is searched, it is possible to easily find out all ATC codes of Chinese names containing “metronidazole,” for example, the result is: metronidazole, A01AB17; Lansoprazole, amoxicillin and metronidazole, A02BD03, etc. Accordingly, the code and the ingredient corresponding to the code can be easily obtained through the search engine software.

Further, it is possible to set the number of ATC codes returned by the search engine software. All of the drug ingredients in the key information of the drug obtained in step 202 are placed in the code inverted index to find a related code or an ingredient corresponding to the code. For each kind of drug ingredient, a maximum return code may be set, or the number of ingredients corresponding to the code may be set to n (n>1), for example, n is set to 10.

Step 204, screening the at least one code based on the key information of the drug and the ingredient corresponding to each code to obtain an anatomical therapeutic chemical classification system code of the drug.

In this embodiment, alternatively, the at least one code may refer to one or more codes. Therefore, after the at least one code is obtained, the number of the at least one code may be first detected. When the at least one code refers to one code, the obtained code is the ATC code. When the at least one code refers to more than one code, it is required to screen the at least one code based on the key information of the drug and the ingredient corresponding to each code to obtain the ATC code of the drug.

The code obtained directly through the retrieval for the inverted index does not necessarily fully meet the requirement for the drug ingredient in the key information of the drug. Therefore, in some alternative implementations of this embodiment, the screening the at least one code based on the key information of the drug and the ingredient corresponding to the each code to obtain an anatomical therapeutic chemical classification system code of the drug includes: detecting, for each code in the at least one code, whether an ingredient corresponding to the code matches the drug ingredient; obtaining a preliminarily screened candidate code including the code, in response to determining that the ingredient corresponding to the code matches the drug ingredient and detections for all codes are completed; and determining that the preliminarily screened candidate code is the anatomical therapeutic chemical classification system code of the drug, in response to detecting that the preliminarily screened candidate code only refers to one code.

In this alternative implementation, the drug ingredient may be expressed in different languages. When detecting whether the drug ingredient matches the ingredient corresponding to the code, the detection may be performed on the similarity of the contents (Chinese names or English words) of the two. Alternatively, whether the drug ingredient matches the ingredient corresponding to the code may be determined through the diseases the two are adapted to treat. For example, if the drug ingredient and the ingredient corresponding to the code may both treat two or more same diseases, it is determined that the drug ingredient matches the ingredient corresponding to the code. Clearly, whether the drug ingredient matches the ingredient corresponding to the code may be detected by other means, which is not limited.

In this embodiment, the preliminarily screened candidate code includes all codes matching all drug ingredients of the drug in the at least one code, i.e., at least one code, and the ingredient corresponding to this code matches the drug ingredient.

In this alternative implementation, by performing matching on the drug ingredient in the key information of the drug and the ingredient corresponding to each code in the at least one code, the preliminarily screened candidate code includes this code is obtained when a matching condition is satisfied. After the detections for all codes in the at least one code are completed, the number of codes in the preliminarily screened candidate code is determined. When there is just one code, the ATC code is obtained. Therefore, the preliminarily screened candidate code can be obtained only by performing matching on the drug ingredient and an inverted index result, which is simple to implement and easy to operate.

In some other alternative implementations of this embodiment, the key information of the drug further includes a drug indication. The screening the at least one code based on the key information of the drug and the ingredient corresponding to the each code to obtain an anatomical therapeutic chemical classification system code of the drug further includes: determining a disease type corresponding to the drug based on the drug indication, in response to detecting that the preliminarily screened candidate code refers to a plurality of codes; and screening a code corresponding to the disease type from the preliminarily screened candidate code as the anatomical therapeutic chemical classification system code of the drug.

According to the method and apparatus for determining a drug code provided in the embodiment of the present disclosure, the description text of the drug is first acquired; next, the key information of the drug in the description text is extracted; then, the at least one code related to the key information of the drug and the ingredient corresponding to the each code are obtained based on the pre-created code inverted index; and finally, the at least one code is screened based on the key information of the drug and the ingredient corresponding to the each code to obtain the anatomical therapeutic chemical classification system code of the drug. Accordingly, through the pre-created coded inverted index, ATC encoding can be automatically performed on medicines according to the description text of the drug, which solves the problems of the majority of pharmacists in their work and provides basic coding information for the medical information system.

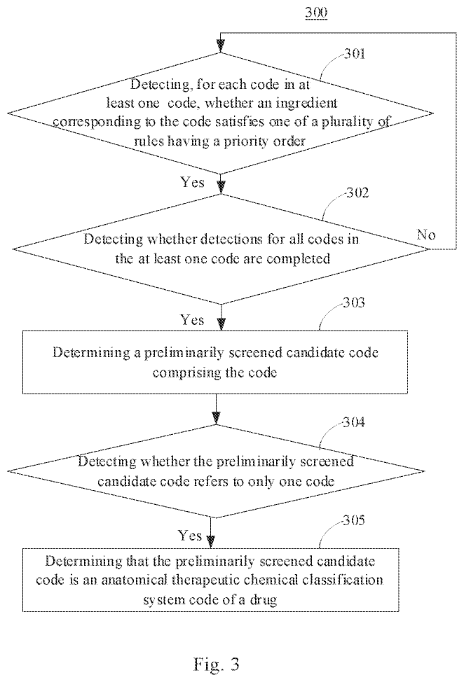

When the key information of the drug includes the drug ingredient, in some alternative implementations of this embodiment, as shown in FIG. 3, FIG. 3 illustrates a flow 300 of an embodiment of a method for obtaining an anatomical therapeutic chemical classification system code of a drug according to the present disclosure. The method for obtaining an anatomical therapeutic chemical classification system code of a drug includes the following steps.

Step 301, detecting, for each code in at least one code, whether an ingredient corresponding to the code satisfies one of a plurality of rules having a priority order; and performing step 302 if the ingredient corresponding to the code satisfies one of the plurality of rules having the priority order.

In this embodiment, the plurality of rules are determined based on drug ingredients. After the ingredient corresponding to the code satisfies any one of the plurality of rules according to the priority order of the rules, the other rules in the plurality of rules may not be taken into consideration any more.

Specifically, the plurality of rules are arranged in a descending order of priorities as follows: (1) the ingredient corresponding to the code includes all drug ingredients of the drug when there are two or more kinds of drug ingredients; (2) the ingredient corresponding to the code includes at least one drug ingredient of the drug and contains a word “compound” when there are two or more kinds of drug ingredients; (3) the ingredient corresponding to the code includes at least one drug ingredient of the drug and does not contain a word “compound” when there are two or more kinds of drug ingredients; and (4) the ingredient corresponding to the code includes the drug ingredient when there is one kind of drug ingredient.

It should be noted that the contents, priority order and number of the rules in the plurality of rules may be adaptively adjusted based on the drug ingredient in the description text of the drug. For example, for a description text that is a description text of a single drug, the plurality of rules may include only the above rules (1) and (4). For example, for a description text that is a description text of a compound drug, the plurality of rules may include only the above rules (1)-(3).

In this alternative implementation, the plurality of rules having the priority order may be applicable to the single drug and the compound drug, and the compound drug is preferentially considered, thus improving the reliability and the comprehensiveness of the ingredient check corresponding to the code.

Step 302, detecting whether detections for all codes in the at least one code are completed; performing step 303 if the detections are completed; and returning to perform step 301 if a detection for at least one code is not completed.

In this embodiment, the code refers to the codes arranged in an order in the at least one code, and is also a current code. In step 302, if the current code (the code) satisfies one of the plurality of rules, the code will be placed in the preliminarily screened candidate code. In step 302, if the current code does not satisfy any one of the plurality of rules, the code will be discarded, and step 301 will be repeated, then an adjacent code behind the current code in the at least one code is used as a current code to perform the detection again.

Step 303, determining a preliminarily screened candidate code including the code, and then performing step 304.

In this embodiment, the preliminarily screened candidate code refers to an ATC code obtained for the first time and satisfying the requirement of the description text of the drug. The ingredient corresponding to each code in the preliminarily screened candidate code satisfies one of the plurality of rules having the priority order. The preliminarily screened candidate code may refer to only one code or a plurality of codes.

In this alternative implementation, the preliminarily screened candidate code includes all codes satisfying one of the plurality of rules in the at least one code, i.e. at least one this code, and the ingredient corresponding to this code satisfies one of the plurality of rules having the priority order.

Step 304, detecting whether the preliminarily screened candidate code refers to only one code; and performing step 305 if a detection result is “only one.”

In this embodiment, when the detection result is “only one code,” it is determined that the current preliminarily screened candidate code is the ATC code of the drug without any subsequent detection.

In this alternative embodiment, for the situation where the preliminarily screened candidate code refers to a plurality of codes, alternatively, similarity matching may be performed on the codes in the preliminarily screened candidate codes, one of a plurality of preliminarily screened candidate codes that has most similarities is used as the ATC code of the drug.

Alternatively, for a description text that is a description text of a compound drug, a preliminarily screened candidate code corresponding to an ingredient containing a word “compound” in all the preliminarily screened candidate codes may be used as the ATC code of the drug. For the description text that is the description text of the compound drug, a preliminarily screened candidate code corresponding to an ingredient not containing the word “compound” in all the preliminarily screened candidate codes may be used as the ATC code of the drug.

Step 305, determining that the preliminarily screened candidate code is an anatomical therapeutic chemical classification system code of a drug.

In this alternative implementation, when the key information of the drug includes the drug ingredient, the anatomical therapeutic chemical classification system code of the drug is determined based on the plurality of rules corresponding to the drug ingredient, which improves the reliability of determining the ATC code.

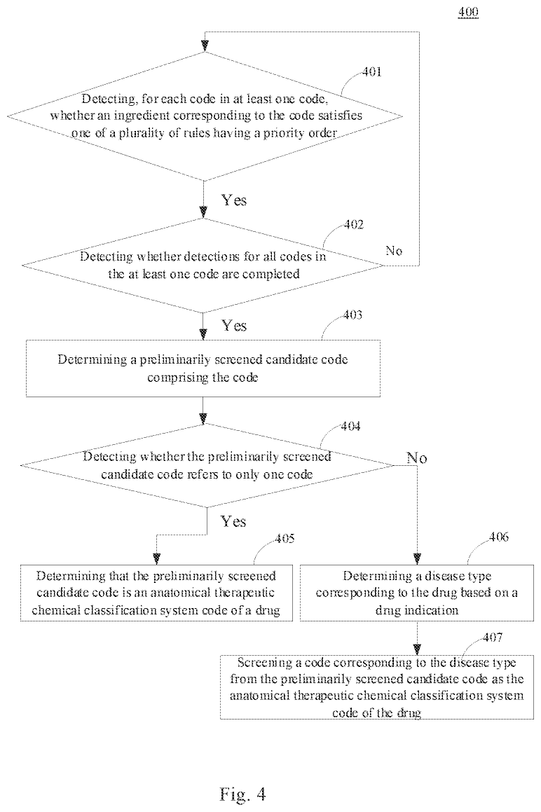

When the key information of the drug includes a drug ingredient and a drug indication, in some alternative implementations of this embodiment, as shown in FIG. 4, FIG. 4 illustrates a flow 400 of another embodiment of the method for obtaining an anatomical therapeutic chemical classification system code of a drug according to the present disclosure. The method for obtaining an anatomical therapeutic chemical classification system code of a drug includes the following steps.

Step 401, detecting, for each code in at least one code, whether an ingredient corresponding to the code satisfies one of a plurality of rules having a priority order; and performing step 402 if the ingredient corresponding to the code satisfies one of the plurality of rules having the priority order.

Step 402, detecting whether detections for all codes in the at least one code are completed; performing step 403 if the detections are completed; and returning to perform step 401 if a detection for at least one code is not completed.

Step 403, determining a preliminarily screened candidate code including the code, and then performing step 404.

Step 404, detecting whether the preliminarily screened candidate code refers to only one code, performing step 405 if a detection result is “only one code,” and performing step 406 if the detection result is that the preliminarily screened candidate code refers to a plurality of codes.

Step 405, determining that the preliminarily screened candidate code is an anatomical therapeutic chemical classification system code of a drug.

It should be understood that the operations and features in steps 401-405 respectively correspond to the operations and features in steps 301-305. Therefore, the descriptions for the operations and features in steps 301-305 are also applicable to steps 401-405, and thus will not be repeated here.

Step 406, determining a disease type corresponding to the drug based on a drug indication, and then performing step 407.

In this embodiment, alternatively, a table of corresponding relationships between indications and disease types may be preset. After the drug indication is obtained, the disease type corresponding to the drug indication can be quickly obtained based on the preset table of the corresponding relationships between the indications and the disease types.

In some alternative implementations of this embodiment, the determining a disease type corresponding to the drug based on a drug indication includes: performing a disease classification on the indication using a pre-trained classification model to obtain the disease type outputted by the classification model.

In practical applications, the classification model may be constructed using a BERT model, such that the classification model performs the disease classification on the indication in the description text of the drug, to obtain probability values of different disease types outputted by the model, for example, classifications of 14 disease types. For example, the indication in the description includes “used for ordinary acne, and can also be used for seborrheic dermatitis, acne rosacea, and folliculitis,” and classifications of 14 disease types are performed to determine which disease the drug is used to treat. These classifications are for digestive system, metabolic system, blood and hematopoietic organs, cardiovascular system, dermatoses, urogenital system, sex hormone, anti-infection, anti-tumor and immunotherapy, musculoskeletal system, nervous system, anti-parasite, respiratory system and sensory system, a total of 14 disease types. These 14 classifications also correspond to the 14 disease types at the first level of the ATC.

For the indication “used for ordinary acne, and can also be used for seborrheic dermatitis, acne rosacea, and folliculitis” in the description, the classification model outputs the respective confidence level scores for the above 14 disease types. For example, for the above indication, the classification scores outputted by the classification model are respectively: digestive system (2%), metabolic system (7%), blood and hematopoietic organs (8%), cardiovascular system (5%), dermatoses (80%), urogenital system (1%), sex hormone (8%), anti-infection (10%), anti-tumor and immunotherapy (2%), musculoskeletal system (2%), nervous system (2%), anti-parasite (2%), respiratory system (2%) and sensory system (2%), and accordingly, the obtained result is that the disease type of the above indication belongs to the dermatoses.

By training a classification model in which a disease type corresponds to a drug indication, it is possible to known what disease the drug is used to treat through the indication text in the description of the drug. In this embodiment, the accuracy of performing the classification using the classification model can reach 93% or above.

In this alternative implementation, by inputting the indication extracted from the description text into the pre-trained classification model, the disease type outputted by the classification model can be obtained. Further, by comparing the obtained disease type with the disease type corresponding to each code in the preliminarily screened candidate code, a preferred ATC code corresponding to the drug in the preliminarily screened candidate code can be obtained. Through the classification model, the accuracy of acquiring the disease type can be improved, and the reliability of obtaining the ATC code of the drug is ensured.

Step 407, screening a code corresponding to the disease type from the preliminarily screened candidate code as the anatomical therapeutic chemical classification system code of the drug.

In this embodiment, there may be one or more disease types. When there is one disease type, the preliminarily screened candidate code corresponding to the disease type is the ATC code of the drug. When there are a plurality of disease types, alternatively, a preliminarily screened candidate code corresponding to a largest number of disease types in the disease types can be used as the ATC code of the drug. Clearly, a code corresponding to an obtained first disease type can alternatively be selected as the ATC code of the drug. The present disclosure is not limited thereto.

In this alternative implementation, when the key information of the drug includes the drug ingredient and the drug indication, the preliminarily screened candidate code in the at least one code is determined based on the drug ingredient. When the preliminarily screened candidate code refers to a plurality of codes, the disease type corresponding to the drug is determined based on the drug indication, and the ATC code of the drug is determined from the preliminarily screened candidate code, which solves the problem that the same drug has a plurality of ATC codes, thereby ensuring the accuracy of determining the ATC code.

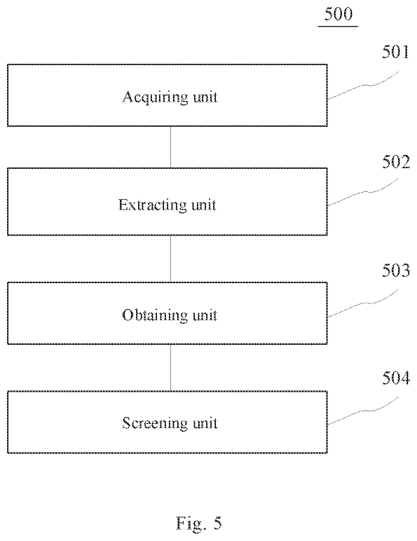

Further referring to FIG. 5, as an implementation of the method shown in the above drawings, the present disclosure provides an embodiment of an apparatus for determining a drug code. The embodiment of the apparatus corresponds to the embodiment of the method shown in FIG. 2. The apparatus may be applied in various electronic devices.

As shown in FIG. 5, the embodiment of the present disclosure provides an apparatus 500 for determining a drug code. The apparatus 500 includes: an acquiring unit 501, an extracting unit 502, an obtaining unit 503 and a screening unit 504. Here, the acquiring unit 501 may be configured to acquire a description text of a drug. The extracting unit 502 may be configured to extract key information of the drug in the description text. The obtaining unit 503 may be configured to obtain at least one code related to the key information of the drug and an ingredient corresponding to each code based on a pre-created code inverted index. The screening unit 504 may be configured to screen the at least one code based on the key information of the drug and the ingredient corresponding to each code to obtain an anatomical therapeutic chemical classification system code of the drug.

In this embodiment, for specific processes of the acquiring unit 501, the extracting unit 502, the obtaining unit 503 and the screening unit 504 in the apparatus 500 for determining a drug code, and their technical effects, reference may be respectively made to step 201, step 202, step 203 and step 204 in the corresponding embodiment of FIG. 2.

In some embodiments, the key information of the drug includes a drug ingredient, and the screening unit 504 includes: a detecting module (not shown in the figure), a preliminarily screening module (not shown in the figure) and a determining module (not shown in the figure). Here, the detecting module may be configured to detect, for each code in the at least one code, whether an ingredient corresponding to the code satisfies one of a plurality of rules having a priority order, the plurality of rules being determined based on the drug ingredient. The preliminarily screening module may be configured to determine a preliminarily screened candidate code including the code, in response to determining that the ingredient corresponding to the code satisfies one of the plurality of rules and detections for all codes are completed. The determining module may be configured to determine that the preliminarily screened candidate code is the anatomical therapeutic chemical classification system code of the drug, in response to detecting that the preliminarily screened candidate code refers to only one code.

In some embodiments, the plurality of rules are arranged in a descending order of priorities as follows: (1) the ingredient corresponding to the code includes all drug ingredients of the drug when there are two or more kinds of drug ingredients; (2) the ingredient corresponding to the code includes at least one drug ingredient of the drug and contains a word “compound” when there are two or more kinds of drug ingredients; (3) the ingredient corresponding to the code includes at least one drug ingredient of the drug and does not contain a word “compound” when there are two or more kinds of drug ingredients; and (4) the ingredient corresponding to the code includes the drug ingredient when there is one kind of drug ingredient.

In some embodiments, the key information of the drug includes a drug ingredient, the screening unit 504 includes: a matching module (not shown in the figure), a responding module (not shown in the figure) and an encoding module (not shown in the figure). Here, the matching module may be configured to detect, for each code in the at least one code, whether an ingredient corresponding to the code matches the drug ingredient. The responding module may be configured to obtain a preliminarily screened candidate code including the code, in response to determining that the ingredient corresponding to the code matches the drug ingredient and detections for all codes are completed. The encoding module may be configured to determine that the preliminarily screened candidate code is the anatomical therapeutic chemical classification system code of the drug, in response to detecting that the preliminarily screened candidate code refers to only one code.

In some embodiments, the key information of the drug further includes a drug indication, the screening unit 504 includes: a classifying module (not shown in the figure) and a confirming module (not shown in the figure). Here, the classifying module may be configured to determine a disease type corresponding to the drug based on the drug indication, in response to detecting that the preliminarily screened candidate code refers to a plurality of codes. The confirming module may be configured to screen a code corresponding to the disease type from the preliminarily screened candidate code as the anatomical therapeutic chemical classification system code of the drug.

In some embodiments, the classifying module may be further configured to performing a disease classification on the indication using a pre-trained classification model to obtain the disease type outputted by the classification model.

According to the apparatus for determining a drug code provided in the embodiment of the present disclosure, the acquiring unit 501 first acquires the description text of the drug; next, the extracting unit 502 extracts the key information of the drug in the description text; then, the obtaining unit 503 obtains the at least one code related to the key information of the drug and the ingredient corresponding to the each code based on the pre-created code inverted index; and finally, the screening unit 504 screens the at least one code based on the key information of the drug and the ingredient corresponding to each code to obtain the anatomical therapeutic chemical classification system code of the drug. Accordingly, through the pre-created coded inverted index, ATC encoding can be automatically performed on medicines according to the description text of the drug, which solves the problems of the majority of pharmacists in their work and provides basic coding information for the medical information system.

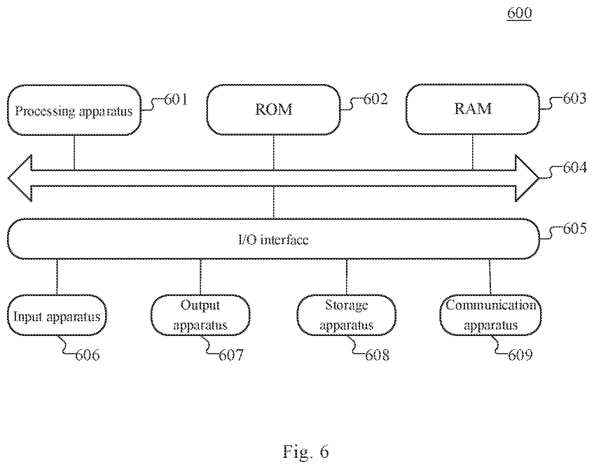

Referring to FIG. 6, FIG. 6 is a schematic structural diagram of an electronic device 600 adapted to implement embodiments of the present disclosure.

As shown in FIG. 6, the electronic device 600 may include a processing apparatus (e.g., a central processing unit and a graphics processing unit) 601, which may execute various appropriate actions and processes in accordance with a program stored in a read-only memory (ROM) 602 or a program loaded into a random access memory (RAM) 603 from a storage apparatus 608. The RAM 603 further stores various programs and data required by operations of the electronic device 600. The processing apparatus 601, the ROM 602 and the RAM 603 are connected to each other through a bus 604. An input/output (I/O) interface 605 is also connected to the bus 604.

Generally, the following components are connected to the I/O interface 605: an input apparatus 606 including a touch screen, a touch tablet, a keyboard, a mouse, etc.; an output apparatus 607 including such as a liquid crystal display device (LCD), a speaker, a vibrator, etc.; a storage apparatus 608 including a tape, a hard disk and the like; and a communication apparatus 609. The communication apparatus 609 may allow the electronic device 600 to communicate wirelessly or wired with other devices to exchange data. Although FIG. 6 shows electronic device 600 with various apparatus, it should be understood that it is not required to implement or have all of the apparatus shown. It may be implemented or have more or fewer apparatus instead. Each box shown in FIG. 6 may represent a single apparatus or multiple apparatus as needed.

In particular, according to the embodiments of the present disclosure, the process described above with reference to the flow chart may be implemented in a computer software program. For example, an embodiment of the present disclosure includes a computer program product, which includes a computer program that is tangibly embedded in a computer-readable medium. The computer program includes program codes for performing the method as illustrated in the flow chart. In such an embodiment, the computer program may be downloaded and installed from a network via the communication apparatus 609, or may be installed from the storage apparatus 608, or may be installed from the ROM 602. The computer program, when executed by the processing apparatus 601, implements the above-mentioned functionalities as defined by the method of the present disclosure.

It should be noted that the computer readable medium in the present disclosure may be computer readable signal medium or computer readable storage medium or any combination of the above two. An example of the computer readable storage medium may include, but not limited to: electric, magnetic, optical, electromagnetic, infrared, or semiconductor systems, apparatus, elements, or a combination of any of the above. A more specific example of the computer readable storage medium may include but is not limited to: electrical connection with one or more wire, a portable computer disk, a hard disk, a random access memory (RAM), a read only memory (ROM), an erasable programmable read only memory (EPROM or flash memory), a fiber, a portable compact disk read only memory (CD-ROM), an optical memory, a magnet memory or any suitable combination of the above. In the present disclosure, the computer readable storage medium may be any physical medium containing or storing programs which may be used by a command execution system, apparatus or element or incorporated thereto. In the present disclosure, the computer readable signal medium may include data signal in the base band or propagating as parts of a carrier, in which computer readable program codes are carried. The propagating data signal may take various forms, including but not limited to: an electromagnetic signal, an optical signal or any suitable combination of the above. The signal medium that can be read by computer may be any computer readable medium except for the computer readable storage medium. The computer readable medium is capable of transmitting, propagating or transferring programs for use by, or used in combination with, a command execution system, apparatus or element. The program codes contained on the computer readable medium may be transmitted with any suitable medium including but not limited to: wireless, wired, optical cable, RF medium etc., or any suitable combination of the above.

The computer readable medium mentioned above may be included in the server, or exist separately without being assembled into the server. The computer readable medium carries one or more programs, and the one or more programs, when executed by the server, cause the server to: acquire a description text of a drug; extract key information of the drug in the description text; obtain at least one code related to the key information of the drug and an ingredient corresponding to each code based on a pre-created code inverted index; and screen the at least one code based on the key information of the drug and the ingredient corresponding to each code to obtain an anatomical therapeutic chemical classification system code of the drug.

A computer program code for performing operations in the present disclosure may be compiled using one or more programming languages or combinations thereof. The programming languages include object-oriented programming languages, such as Java, Smalltalk or C++, and also include conventional procedural programming languages, such as “C” language or similar programming languages. The program code may be completely executed on a user's computer, partially executed on a user's computer, executed as a separate software package, partially executed on a user's computer and partially executed on a remote computer, or completely executed on a remote computer or server. In the circumstance involving a remote computer, the remote computer may be connected to a user's computer through any network, including local area network (LAN) or wide area network (WAN), or may be connected to an external computer (for example, connected through Internet using an Internet service provider).

The flow charts and block diagrams in the accompanying drawings illustrate architectures, functions and operations that may be implemented according to the systems, methods and computer program products of the various embodiments of the present disclosure. In this regard, each of the blocks in the flow charts or block diagrams may represent a module, a program segment, or a code portion, said module, program segment, or code portion including one or more executable instructions for implementing specified logic functions. It should also be noted that, in some alternative implementations, the functions denoted by the blocks may occur in a sequence different from the sequences shown in the accompanying drawings. For example, any two blocks presented in succession may be executed, substantially in parallel, or they may sometimes be in a reverse sequence, depending on the function involved. It should also be noted that each block in the block diagrams and/or flow charts as well as a combination of blocks may be implemented using a dedicated hardware-based system performing specified functions or operations, or by a combination of a dedicated hardware and computer instructions.

The described units involved in the embodiments of the present disclosure may be implemented by means of software or hardware. The described units may also be provided in a processor. For example, the processor may be described as: a processor including an acquiring unit, an extracting unit, an obtaining unit and a screening unit. Here, the names of these units do not in some cases constitute a limitation to such units themselves. For example, the acquiring unit may alternatively be described as “a unit for acquiring a description text of a drug.

The above description only provides an explanation of the preferred embodiments of the present disclosure and the technical principles used. It should be appreciated by those skilled in the art that the inventive scope of the present disclosure is not limited to the technical solutions formed by the particular combinations of the above-described technical features. The inventive scope should also cover other technical solutions formed by any combinations of the above-described technical features or equivalent features thereof without departing from the concept of the present disclosure. Technical schemes formed by the above-described features being interchanged with, but not limited to, technical features with similar functions disclosed in the present disclosure are examples.

1. A method for determining a drug code, comprising:
acquiring a description text of a drug;
extracting key information of the drug in the description text;
obtaining at least one code related to the key information of the drug and an ingredient corresponding to each code based on a pre-created code inverted index; and
screening the at least one code based on the key information of the drug and the ingredient corresponding to each code to obtain an anatomical therapeutic chemical classification system code of the drug.

2. The method according to claim 1, wherein the key information of the drug comprises a drug ingredient, and
screening the at least one code based on the key information of the drug and the ingredient corresponding to each code to obtain the anatomical therapeutic chemical classification system code of the drug comprises:
detecting, for each code in the at least one code, whether an ingredient corresponding to the code satisfies one of a plurality of rules having a priority order, the plurality of rules being determined based on the drug ingredient;
determining a preliminarily screened candidate code comprising the code, in response to determining that the ingredient corresponding to the code satisfies one of the plurality of rules and detections for all codes are completed; and
determining that the preliminarily screened candidate code is the anatomical therapeutic chemical classification system code of the drug, in response to detecting that the preliminarily screened candidate code refers to only one code.

3. The method according to claim 2, wherein the plurality of rules are arranged in a descending order of priorities as follows:
(1) the ingredient corresponding to the code comprises all drug ingredients of the drug when there are two or more kinds of drug ingredients;
(2) the ingredient corresponding to the code comprises at least one drug ingredient of the drug and contains a word “compound” when there are two or more kinds of drug ingredients;
(3) the ingredient corresponding to the code comprises at least one drug ingredient of the drug and does not contain the word “compound” when there are two or more kinds of drug ingredients; and
(4) the ingredient corresponding to the code comprises the drug ingredient when there is one kind of drug ingredient.

4. The method according to claim 1, wherein the key information of the drug comprises a drug ingredient, and
screening the at least one code based on the key information of the drug and the ingredient corresponding to each code to obtain the anatomical therapeutic chemical classification system code of the drug comprises:
detecting, for each code in the at least one code, whether an ingredient corresponding to the code matches the drug ingredient;
obtaining a preliminarily screened candidate code comprising the code, in response to determining that the ingredient corresponding to the code matches the drug ingredient and detections for all codes are completed; and
determining that the preliminarily screened candidate code is the anatomical therapeutic chemical classification system code of the drug, in response to detecting that the preliminarily screened candidate code refers to only one code.

5. The method according to claim 2, wherein the key information of the drug further comprises a drug indication, and
screening the at least one code based on the key information of the drug and the ingredient corresponding to each code to obtain the anatomical therapeutic chemical classification system code of the drug further comprises:
determining a disease type corresponding to the drug based on the drug indication, in response to detecting that the preliminarily screened candidate code refers to a plurality of codes; and
screening a code corresponding to the disease type from the preliminarily screened candidate code as the anatomical therapeutic chemical classification system code of the drug.

6. The method according to claim 5, wherein determining the disease type corresponding to the drug based on the drug indication comprises:
performing a disease classification on the indication using a pre-trained classification model to obtain the disease type outputted by the classification model.

7. An apparatus for determining a drug code, comprising:
one or more processors; and
a memory storing one or more programs;
wherein the one or more programs, when executed by the one or more processors, cause the one or more processors to perform operations, the operations comprising:
acquiring a description text of a drug;
extracting key information of the drug in the description text;
obtaining at least one code related to the key information of the drug and an ingredient corresponding to each code based on a pre-created code inverted index; and
screening the at least one code based on the key information of the drug and the ingredient corresponding to each code to obtain an anatomical therapeutic chemical classification system code of the drug.

8. The apparatus according to claim 7, wherein the key information of the drug comprises a drug ingredient, and screening the at least one code based on the key information of the drug and the ingredient corresponding to each code to obtain the anatomical therapeutic chemical classification system code of the drug:
detecting, for each code in the at least one code, whether an ingredient corresponding to the code satisfies one of a plurality of rules having a priority order, the plurality of rules being determined based on the drug ingredient;
determining a preliminarily screened candidate code comprising the code, in response to determining that the ingredient corresponding to the code satisfies one of the plurality of rules and detections for all codes are completed; and
determining that the preliminarily screened candidate code is the anatomical therapeutic chemical classification system code of the drug, in response to detecting that the preliminarily screened candidate code refers to only one code.

9. The apparatus according to claim 8, wherein the key information of the drug further comprises a drug indication, and
screening the at least one code based on the key information of the drug and the ingredient corresponding to each code to obtain the anatomical therapeutic chemical classification system code of the drug further comprises:
determining a disease type corresponding to the drug based on the drug indication, in response to detecting that the preliminarily screened candidate code refers to a plurality of codes; and
screening a code corresponding to the disease type from the preliminarily screened candidate code as the anatomical therapeutic chemical classification system code of the drug.

10. (canceled)

11. A non-transitory computer readable medium, storing a computer program, wherein the program, when executed by a processor, causes the processor to perform operations, the operations comprising:
acquiring a description text of a drug;
extracting key information of the drug in the description text;
obtaining at least one code related to the key information of the drug and an ingredient corresponding to each code based on a pre-created code inverted index; and
screening the at least one code based on the key information of the drug and the ingredient corresponding to each code to obtain an anatomical therapeutic chemical classification system code of the drug.

12. (canceled)

13. The non-transitory computer readable medium according to claim 11, wherein the key information of the drug comprises a drug ingredient, and
screening the at least one code based on the key information of the drug and the ingredient corresponding to each code to obtain the anatomical therapeutic chemical classification system code of the drug comprises:
detecting, for each code in the at least one code, whether an ingredient corresponding to the code satisfies one of a plurality of rules having a priority order, the plurality of rules being determined based on the drug ingredient;
determining a preliminarily screened candidate code comprising the code, in response to determining that the ingredient corresponding to the code satisfies one of the plurality of rules and detections for all codes are completed; and
determining that the preliminarily screened candidate code is the anatomical therapeutic chemical classification system code of the drug, in response to detecting that the preliminarily screened candidate code refers to only one code.

14. The non-transitory computer readable medium according to claim 13, wherein the plurality of rules are arranged in a descending order of priorities as follows:
(1) the ingredient corresponding to the code comprises all drug ingredients of the drug when there are two or more kinds of drug ingredients;
(2) the ingredient corresponding to the code comprises at least one drug ingredient of the drug and contains a word “compound” when there are two or more kinds of drug ingredients;
(3) the ingredient corresponding to the code comprises at least one drug ingredient of the drug and does not contain the word “compound” when there are two or more kinds of drug ingredients; and
(4) the ingredient corresponding to the code comprises the drug ingredient when there is one kind of drug ingredient.

15. The non-transitory computer readable medium according to claim 11, wherein the key information of the drug comprises a drug ingredient, and
screening the at least one code based on the key information of the drug and the ingredient corresponding to each code to obtain the anatomical therapeutic chemical classification system code of the drug comprises:
detecting, for each code in the at least one code, whether an ingredient corresponding to the code matches the drug ingredient;
obtaining a preliminarily screened candidate code comprising the code, in response to determining that the ingredient corresponding to the code matches the drug ingredient and detections for all codes are completed; and
determining that the preliminarily screened candidate code is the anatomical therapeutic chemical classification system code of the drug, in response to detecting that the preliminarily screened candidate code refers to only one code.

16. The non-transitory computer readable medium according to claim 13, wherein the key information of the drug further comprises a drug indication, and
screening the at least one code based on the key information of the drug and the ingredient corresponding to each code to obtain the anatomical therapeutic chemical classification system code of the drug further comprises:
determining a disease type corresponding to the drug based on the drug indication, in response to detecting that the preliminarily screened candidate code refers to a plurality of codes; and
screening a code corresponding to the disease type from the preliminarily screened candidate code as the anatomical therapeutic chemical classification system code of the drug.

17. The non-transitory computer readable medium according to claim 16, wherein determining the disease type corresponding to the drug based on the drug indication comprises:
performing a disease classification on the indication using a pre-trained classification model to obtain the disease type outputted by the classification model.

18. The apparatus according to claim 8, wherein the plurality of rules are arranged in a descending order of priorities as follows:
(1) the ingredient corresponding to the code comprises all drug ingredients of the drug when there are two or more kinds of drug ingredients;
(2) the ingredient corresponding to the code comprises at least one drug ingredient of the drug and contains a word “compound” when there are two or more kinds of drug ingredients;
(3) the ingredient corresponding to the code comprises at least one drug ingredient of the drug and does not contain the word “compound” when there are two or more kinds of drug ingredients; and
(4) the ingredient corresponding to the code comprises the drug ingredient when there is one kind of drug ingredient.

19. The apparatus according to claim 7, wherein the key information of the drug comprises a drug ingredient, and
screening the at least one code based on the key information of the drug and the ingredient corresponding to each code to obtain the anatomical therapeutic chemical classification system code of the drug comprises:
detecting, for each code in the at least one code, whether an ingredient corresponding to the code matches the drug ingredient;
obtaining a preliminarily screened candidate code comprising the code, in response to determining that the ingredient corresponding to the code matches the drug ingredient and detections for all codes are completed; and
determining that the preliminarily screened candidate code is the anatomical therapeutic chemical classification system code of the drug, in response to detecting that the preliminarily screened candidate code refers to only one code.

20. The apparatus according to claim 9, wherein determining the disease type corresponding to the drug based on the drug indication comprises:
performing a disease classification on the indication using a pre-trained classification model to obtain the disease type outputted by the classification model.
